(19) United States
(12) Patent Application Publication
(10) Pub. No.: US 2022/0338744 A1
(43) Pub. Date: Oct. 27, 2022

(54) OVERALL LOOP SYSTEM

(71) Applicant: CARDIORENAL, Strasbourg (FR)

(72) Inventors: Guilhem HENRION, Strasbourg (FR); Moez KAROUI, Strasbourg (FR); Maurice BERENGER, Strasbourg (FR)

(73) Assignee: CARDIORENAL, Strasbourg (FR)

(21) Appl. No.: 17/763,784

(22) PCT Filed: Sep. 25, 2020
(86) PCT No.: PCT/EP2020/076919

§ 371 Date: Mar. 25, 2022

Related U.S. Application Data
(60) Provisional application No. 62/906,894, filed on Sep. 27, 2019.

(30) Foreign Application Priority Data
Sep. 27, 2019 (EP) 19200164.2

(51) Int. Cl. A61B 5/0205; G16H 50/30; G16H 20/10; A61B 5/00

(52) U.S. Cl. CPC A61B 5/0205; G16H 50/30; G16H 20/10; A61B 5/0022; A61B 5/0006

(57) ABSTRACT

A system and a method for optimizing a treatment regimen of a subject suffering from heart failure with a risk of congestive heart failure or a risk of side effects due to heart failure medication. Also, a computer program product for optimizing a treatment regimen of a user and a computer-readable storage medium, both of which include instructions that when the program is executed by a computer causes the computer to carry out the steps of the method.

### FIELD OF INVENTION

The present invention relates to a system and a method for optimizing a treatment regimen of a user suffering from heart failure or at risk of side effects due to heart failure medication. In particular, the present invention relates to a system and a method for analyzing, at least two physiological parameters in a blood sample of a user.

### BACKGROUND OF INVENTION

Heart failure is defined as the incapacity of the heart to pump blood normally. Different causes can lead to heart failure: evolution of heart infarcts, angina, arterial hypertension (HTA). Heart Failure diagnosis and follow-up can be facilitated by a blood test analyzing improvement or worsening of the user's condition.

Traditionally, the diagnostic and follow-up are carried out in a medical center directly by a practitioner or in a testing laboratory for blood test measurement. Different health management systems or medical point of care exist.

As an example of health management of the prior art, U.S. Pat. No. 10,309,954 reports an assay apparatus comprising at least one assay module; and a portable frame adapted to releasably retain the at least one assay module. The at least one assay module is adapted to perform at least one assay. The assay module comprises a sample receiver and an assay device operatively associated with the sample receiver. In some embodiments, the assay apparatus further comprises at least one functional module releasably retained by the portable frame. The functional module is operatively associated with the assay module when retained by the portable frame.

However, the results are not analyzed by a practitioner. Therefore, an appointment in a medical center to review the result of the analyses is necessary, which is not optimal for responsive optimization of the user's treatment regimen.

As another example of point of care of the prior art, U.S. Pat. No. 8,283,155 reports portable medical devices that allow real-time detection of analytes from a biological fluid. The methods and devices are particularly useful for providing point-of-care testing for a variety of medical applications. However, this invention requires an assay assembly containing reactants and such assay transfers the raw data. The practitioner has to analyze the data and therefore this induces a delay to treat the users who are in an emergency situation and require a fast adaptation for their recommendations and drug prescriptions.

Consequently, there is a need to find a fast, simple, cost-effective, and accurate system and method to optimizing drug regimen by the practitioner with a remote frequent analysis of patient physiological parameters and follow-up.

### SUMMARY

The present invention relates to a system for optimizing a treatment regimen of a subject, said subject suffering of heart failure with a risk of congestive heart failure or a risk of side effects due to heart failure medication, said system comprising:
a reception module configured to receive at least two physiological parameters, of which at least one being a hematological parameter obtained from the analysis of a blood sample of the subject;
a severity estimation module configured to estimate a degree of severity for each physiological parameter by comparing each physiological parameter to at least one predefined threshold and/or range of values;
a subject's history analysis module configured to analyze a subject's medical history wherein the medical history is retrieved from a database, said medical history comprising at least one of the following: clinical markers, the values of the at least two physiological parameters as a function of time, a current prescription of the user, at least one previous prescription and/or information related to former health condition;
a first calculation module configured to perform an analysis of the subject's medical history and the at least two physiological parameters whenever the degrees of severity estimated for each physiological parameter is comprised in a range of values corresponding to a normal condition of the subject, wherein the analysis is performed by providing as input to a decision tree the subject's medical history and the at least two physiological parameters so as to output a recommendation to a user, said recommendation comprising at least one medicament and a dosage of said medicament;
a second calculation module configured to perform an analysis of the subject's medical history and the at least two physiological parameters whenever at least one of the degrees of severity estimated for each physiological parameter falls out of a range of values (R) corresponding to a normal condition of the subject, wherein the second calculation module is configured to output, based on the analysis, a recommendation to the user comprising at least a medicament and a dosage of said medicament.

The present invention allows to provide a recommendation to the user that is adapted to his/her specific health status thanks to the alternative use of the two calculation modules according to the degrees of severity estimated for each physiological parameter.

According to one embodiment, the one hematological parameter is measured daily.

According to one embodiment, the at least one hematological parameter is selected from the following list: hemoglobin, potassium or creatinine.

According to one embodiment, the at least one hematological parameter is the blood natriuretic peptide.

According to one embodiment, the at least one of the physiological parameters is selected from the following list: pulmonary artery pressure, lung impedance, weight, blood oxygen, blood pressure or electrocardiographic signal.

According to one embodiment, the first calculation module being configured to provide three possible outputs:
a recommendation comprising the same medicament and dosage of the current prescription, whenever in a first preceding predefined time period at least one of the physiological values of the subject fall out of the range of values (R) corresponding to a normal condition and/or whenever in a second preceding predefined time period the current prescription has been provided to the subject by the user;
a recommendation comprising the same medicament of the current prescription and an updated dosage;
a recommendation comprising a medicament and a dosage different from the current prescription.

According to one embodiment, when at least one of the degrees of severity estimated for each physiological parameter fall out of the range of values corresponding to a normal condition of the subject, the recommendation outputted by the second calculation module is determined from a matrix configured to associate each combination of values of the physiological parameters to at least one medicament and its dosage.

According to one embodiment, the system further comprises a visualization module configured to display the recommendation together with a graphical representation of each of at least two physiological parameters overtime.

According to one embodiment, the visualization module is further configured to display the current prescription of the user and a link to a medical history of the user.

According to one embodiment, the visualization module further configured to display a dashboard, the dashboard being configured to represent a subject identifier long with module outputs obtained from the subject's history analysis module, the first calculation module and the second calculation module.

According to one embodiment, the dashboard represents simultaneously subject outputs obtained from multiple subjects for the user.

The present invention relates as well to a method for optimizing a treatment regimen of a subject, said subject suffering of heart failure with a risk of congestive heart failure or a risk of side effects due to heart failure medication, said method comprising the following steps:
obtaining at least two physiological parameters, of which at least one being a hematological parameter obtained from the analysis of a blood sample of the subject;
estimating a degree of severity for each physiological parameter by comparing each physiological parameter to at least one predefined threshold and/or at least one predefined range of values;
analyzing a subject's medical history, wherein the medical history is retrieved from a database, said medical history comprising at least one of the following: clinical markers, the values of the at least two physiological parameters as a function of time, a current prescription, at least one previous prescription and/or information related to former health condition;
if the degrees of severity estimated for each physiological parameter is comprised in a range of values corresponding to a normal condition of the subject, providing as input to a decision tree the subject's medical history and the at least two physiological parameters so as to output a recommendation to a user comprising at least a medicament and a dosage of said medicament;
if at least one of the degrees of severity estimated for each physiological parameter is not comprised in a range of values corresponding to a normal condition of the subject, outputting to the user a recommendation comprising at least a medicament and a dosage of said medicament.

According to one embodiment, the at least one hematological parameter is selected from the following list: hemoglobin, potassium or creatinine.

According to one embodiment, the at least one of the physiological parameters is selected from the following list: pulmonary artery pressure, lung impedance, weight, blood oxygen, blood pressure or electroencephalographic signal.

According to one embodiment, the decision tree being configured to provide three possible outputs:
a recommendation comprising the same medicament and dosage of the current prescription, whenever in a first preceding predefined time period at least one of the physiological values of the subject fall out of the range of values (R) corresponding to a normal condition and/or whenever in a second preceding predefined time period the current prescription have been provided to the subject by the user;
a recommendation comprising the same medicament of the current prescription and an updated dosage;
a recommendation comprising a medicament and a dosage different from the current prescription.

According to one embodiment, when at least one of the degrees of severity estimated for each physiological parameter fall out of the range of values corresponding to a normal condition of the subject the recommendation is determined from a matrix configured to associate each combination of values of the physiological parameters to at least one medicament and its dosage.

According to one embodiment, the method further comprises displaying the recommendation together with a graphical representation of each of at least two physiological parameters overtime, the current prescription of the user and a link to a medical history of the user.

According to one embodiment, the method further comprises displaying a dashboard, the dashboard being configured to represent subject outputs obtained from the subject's history analysis module, the first calculation module and the second calculation module.

According to one embodiment, the dashboard represents simultaneously subject outputs obtained from multiple subjects for the user.

Another aspect of the present invention relates to a computer program product for optimizing a treatment regimen of a user, the computer program product comprising instructions which, when the program is executed by a computer, causes the computer to carry out the steps of the method according to any one of the embodiments hereabove.

Yet another aspect of the present invention relates to a computer-readable storage medium comprising instructions which, when the program is executed by a computer, causes the computer to carry out the steps of the method according to any one of the embodiments hereabove.

Another aspect of the present invention relates to a system for optimizing a treatment regimen of a user suffering of heart failure with a risk of congestive heart failure or a risk of side effects due to heart failure medication by analyzing at least two physiological parameters in a blood sample of the user, comprising:
at least two modules, the first module, in the user's location, being configured to acquire at least two parameters from the blood sample and to transfer data to a database, the second module, in a medical center's location, being configured to acquire and transfer data,
a data processing module configured to carry out the steps of:
   analyzing the at least two parameters,
   analyzing the user's medical history wherein the medical history is retrieved from the database, said medical history comprising at least medical record, clinical markers, parameters evolution and parameters history, the current prescription of the user, previous prescription and information related to former health condition,
   generating heart failure medication management recommendation from the computing of the at least two parameters results and the medical history of the user,
   displaying the recommendation together with a graphical representation of each of the at least two parameters overtime, the contents of the at least two parameters resulting of the analysis, the current prescription of the user and a link to a medical history of the user.

In one embodiment, the at least two parameters are hemoglobin and either potassium or creatinine or both potassium and creatinine. The measures of hemoglobin, potassium and creatinine are the main parameters for detecting heart failure worsening or side effects due to heart failure medication.

In a further embodiment, the frequency of the analysis ranges from at least one analysis per month to more than one analysis per day. The frequency of the analysis is defined by a clinician and depends of the condition of the user. In another embodiment, the frequency is variable over time.

In another embodiment shown in FIG. 2, the first module 1 is a user home box comprising:
a port for receiving a stick, said stick being a single use stick configured for analyzing a blood sample of the user, the stick further comprising one end with an electronic connector and at least one microfluidic system for blood parameters measurement.
a processing element configured to analyze the blood sample collected to the stick so as to extract the parameters level,

In another embodiment, the second module further displays raw data, said raw data being output from the processing element of the first module. The raw data can be analyzed by a physician to make his own decision.

In one embodiment, other parameters are measured, such as blood pressure measured with a tensiometer connected to the first module, heart rate measured with a heart rate monitor connected to the first module and/or weight measured with a weight scale connected to the first module. Those parameters allow better understanding of the user's condition.

In another embodiment, the second module comprises a dashboard, the dashboard being configured to display the result of the analysis.

The present invention also relates to a method for optimizing a treatment regimen of a user suffering of heart failure with a risk of congestive heart failure or a risk of side effects due to heart failure medication by analyzing, at least two physiological parameters in a blood sample of the user, said method comprising the following steps:
analyzing the at least two parameters,
analyzing the user's medical history wherein the medical history is retrieved from the database, said medical history comprising at least medical record, clinical markers, parameters evolution and parameters history, the current prescription of the user, previous prescription and information related to former health condition,
generating recommendation from the computing of the at least two parameters results and the medical history of the user, considering at the same time the recommendation, a graphical representation of
each of the at least two parameters overtime, the contents of the at least two parameters resulting of the analysis and a link to a medical history of the user.

The present invention also relates to a computer program product for monitoring physiological parameters in a blood sample, the computer program product comprising instructions which, when the program is executed by a computer, caused the computer to carry out the steps of the method.

The present invention also relates to a computer-readable storage medium comprising instructions which, when the program is executed by a computer, causes the computer to carry out the steps of the method.

### Definitions

In the present invention, the following terms have the following meanings:
“Worsening renal function” refers to the description of the evolution of the renal function from the baseline. Calculated as a % of evolution of creatinine contents from baseline creatinine level.
“Prescription”: refers to a health care program implemented by a physician or other qualified health care practitioner in the form of instructions that govern the plan of care for an individual patient.

### BRIEF DESCRIPTION OF THE DRAWINGS

Features and advantages of the invention will become apparent from the following description of embodiments of a system, this description being given merely by way of example and with reference to the appended drawings in which:

FIG. 1 is a block diagram representing the main steps of the method of the present invention.

FIG. 2 is a schematic representation of the system of the invention according to one embodiment.

### DETAILED DESCRIPTION

The following detailed description will be better understood when read in conjunction with the drawings. For the purpose of illustrating, the system and method are shown in the preferred embodiments. It should be understood, however that the present invention is not limited to the precise arrangements, structures, features, embodiments, and aspect shown. Accordingly, it should be understood that where features mentioned in the appended claims are followed by reference signs, such signs are included solely for the purpose of enhancing the intelligibility of the claims and are in no way limiting on the scope of the claims.

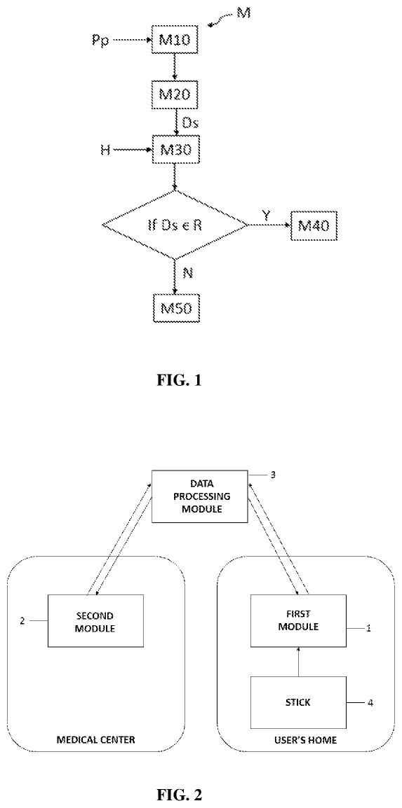

As shown in FIG. 1, the method M of the present invention for optimizing a treatment regimen of a subject comprises a first step M10 of obtaining at least two physiological parameters Pp, of which at least one being a hematological parameter obtained from the analysis of a blood sample of the subject.

In one embodiment, the hematological parameter is selected from the following list: hemoglobin, potassium or creatinine. Advantageously, the hemoglobin, potassium and creatinine are the main parameters for detecting heart failure worsening or side effects due to heart failure medication.

This at least one hematological parameter may be frequently acquired, for example daily or weekly according to medical prescriptions.

According to one embodiment, the at least one physiological parameter, which is not a hematological parameter, is selected from the following list: pulmonary artery pressure, lung impedance, weight, blood oxygen, blood pressure or electrocardiographic signal.

Preferably, the value of one physiological parameter is received with the time and date of the measurement.

This physiological parameter contributes all to the evaluation of the health status of a patient suffering of heart failure with a risk of congestive heart failure or a risk of side effects due to heart failure medication. Indeed, unintentional weight loss is a serious complication associated to heart failure, especially in patients with severe heart failure. Weight loss concerns a large percentage of heart failure patients and it have been observed that once patients start losing weight, survival rates drop significantly. Concerning, pulmonary artery pressure, pulmonary hypertension may cause heart disfunction that may result in heart failure. Indeed, heart failure is the most common cause of death in patients who suffer from pulmonary hypertension. Low oxygen saturation levels are characteristic of heart failure due to the heart's inability to receive oxygen-rich blood from the lungs. Hypertension (i.e. high blood pressure) is a leading cause of cardiovascular disease, stroke, and death. Analysis of electrocardiographic signal allow to directly visualize the electrical activity of the heart and is used to make a preliminary evaluation of patient suffering of heart failure.

The physiological parameter may be as well a signal measured by a cardiac resynchronization device or a pacemaker.

According to one embodiment, the method further comprises analyzing the physiological parameters (i.e.: hemoglobin, potassium, creatinine, etc.) so as to obtain at least one computed physiological parameter (i.e. Estimated Glomerular Filtration Rate, Worsening renal function, etc.).

According to one embodiment, the method further comprises the step M 20 estimating a degree of severity Ds for each physiological parameter by comparing each physiological parameter Pp to at least one predefined threshold and/or at least one predefined range of values. The degree of severity Ds may be calculated taking into account the last 3, 4, 5 or more measurements received for each physiological parameter Pp. For example, a level of potassium in the last blood measurement inferior to 3 mmol/L is associated to a severe hypokalemia (i.e. low level of potassium (K+) in the blood serum) or a level of potassium in the last two blood measurement inferior to 3.5 mmol/L is associated to a moderate hypokalemia. This kind of predefined threshold and/or at least one predefined range are obtained from the medical indication comprised in medical guidelines.

For the estimation of the degree of severity Ds may be used as well at least one computed physiological parameter.

The evolution of the potassium parament in time may be used to evaluate the degree of severity Ds of kalemia (i.e. level of potassium in the blood serum), in terms of severe, mild, moderate hyperkalemia or hypokalemia, or normal kalemia. The hemoglobin parameter is used to estimate the degree of severity Ds of congestion in terms of augmentation, diminution of congestion or no significative congestive status variation. Finally, the creatinine parameter is used to estimate the degree of severity Ds of the worsening of the renal function in terms of mild, moderate or severe worsening or no significative worsening of the renal function.

In one embodiment, the method comprises a step M30 of analyzing a subject's medical history H. The medical history H is generally retrieved from a database, notably a medical database. Said medical history H comprising at least one of the following: clinical markers, the values of the at least two physiological parameters Pp as a function of time, a current prescription, at least one previous prescription and/or information related to former health condition. Indeed, the information of the database may be continuously update to store the values of the physiological parameters measured for the patients. The medical history H may as well comprise a value baseline for each physiological parameters Pp set by the system by default or by the physician. The medical history H may further comprise the target dosage and therapy for heart failure treatment (define by default according to learned societies or by the physician) and the history of output obtained from using the method of the present invention for one patient. The current prescription may comprise a dosage, frequency and DCI of each treatment. The clinical markers may include at least gender or size or age.

According to one embodiment, the step M30 of analyzing a subject's medical history H is configured to analyze subject's medical history H to compute windows of therapy optimization.

According to one embodiment, the method is configured to compere the degrees of severity Ds estimated for each physiological parameter Pp to a range of values R corresponding to a normal condition of the subject in order to select the next step to be implemented, M40 or M50. This step is comparison is represented by the decision block (i.e. turbot block) in the block diagram of FIG. 1. The range of values may be established using known medical guidelines.

According to one embodiment, whenever the degrees of severity estimated for each physiological parameter Pp is comprised in the range of values R corresponding to a normal condition of the subject, the method implements the step M40 of providing as input to a decision tree the subject's medical history H and the at least two physiological parameters Pp so as to output a recommendation to a user comprising at least a medicament and a dosage of said medicament.

A decision tree is a decision support tool that uses a tree-like model of decisions and their possible consequences and contains conditional control statements. Decision trees are supervised learning method that advantageously allow to accurately map non-linear relationship. Notably, a decision tree comprises internal nodes, representing condition, based on which the tree splits into branches (i.e. edges). The end of a branch that doesn't split anymore is a leaf representing the decision which is the output of the decision tree.

According to one embodiment, the decision tree being configured to provide three possible outputs.

According to one embodiment, one of the three output is a recommendation comprising the same medicament and dosage of the current prescription, whenever in a first preceding predefined time period at least one of the physiological values of the subject fall out of the range of values R corresponding to a normal condition and/or whenever in a second preceding predefined time period the current prescription have been provided to the subject by the user.

According to one embodiment, one of the three output is a recommendation comprising the same medicament of the current prescription and an updated dosage.

According to one embodiment, one of the three output is a recommendation comprising a medicament and a dosage different from the current prescription.

According to one embodiment, if at least one of the degrees of severity Ds estimated for each physiological parameter Pp is not comprised in a range of values R corresponding to a normal condition of the subject, the method is configured to implement the step M50 of outputting to the user a recommendation comprising at least a medicament and a dosage of said medicament.

According to one embodiment, the recommendation further lists all the action that the physician can perform to mitigate the detected situation and eliminate worsening situation due to extra Heart failure causes.

In one embodiment, when at least one of the degrees of severity estimated for each physiological parameter Pp fall out of the range of values R corresponding to a normal condition of the subject the recommendation is determined from a matrix configured to associate each combination of values of the physiological parameters to at least one medicament and its dosage.

The recommendations output of the present method may be presented to the user (i.e. physician) on a screen.

In one embodiment, the recommendations are used to classify the patients and present them on a dashboard by categories on a screen. Each category appears based on an emergency criterion of the patient.

According to one embodiment, the method comprises displaying the recommendation together with a graphical representation of each of at least two physiological parameters Pp overtime, the current prescription of the user and a link to a medical history of the user.

According to one embodiment, the method further comprises displaying a dashboard, the dashboard being configured to represent subject outputs obtained from the subject's history analysis module, the first calculation module and the second calculation module.

In one embodiment, the dashboard represents simultaneously subject outputs obtained from multiple subjects for the user.

The present invention also relates to the system for optimizing a treatment regimen of a subject, said subject suffering of heart failure with a risk of congestive heart failure or a risk of side effects due to heart failure medication. This system is configured to implement the steps of the method of the present invention.

The system of the present invention aims to help and guide the physician through this complex analysis by providing: triage of the patient, and a simple and straightforward recommendation for the adaptation of the therapy. The physician will be able to adjust and optimize the heart failure patient therapy in a personalized way according to its physiological parameters' evolution.

According to one embodiment, the system comprises a reception module configured to receive at least two physiological parameters Pp, of which at least one being a hematological parameter obtained from the analysis of a blood sample of the subject. The at least two physiological parameters Pp may be received from a data storage medium by means of wire or wireless connection. Notably, the physiological parameters one acquired may be stored on a medical database to which the system may have access.

The system may have a two modules structure wherein a first module in possession of the user is configured to acquire the hematological parameter from the blood sample and to transfer data to a database; and a second module, located in a medical center's, is configured to receive, acquire and transfer data.

The first module may be a user home box comprising:
a port for receiving a stick, said stick being a single use stick configured for analyzing a blood sample of the user, the stick further comprising one end with an electronic connector and at least one microfluidic system for blood parameters measurement, and
a processing element configured to analyze the blood sample collected to the stick so as to extract the hematological parameters values.

According to one embodiment, the system comprises a severity estimation module configured to estimate a degree of severity Ds for each physiological parameter Pp by comparing each physiological parameter Pp to at least one predefined threshold and/or range of values. The threshold ana/or ranges may be established on the base of data in medical guideline, or inputted by a user or selected by an automated optimization algorithm configured to establish the optimal degree of severity Ds class base on physiological parameter Pp as input.

According to one embodiment, the system comprises a subject's history analysis module configured to analyze a subject's medical history wherein the medical history is retrieved from a database, said medical history comprising at least one of the following: clinical markers, the values of the at least two physiological parameters as a function of time, a current prescription of the user, at least one previous prescription and/or information related to former health condition. The clinical markers may include at least gender or size or age.

According to one embodiment, the system comprises a first calculation module configured to perform an analysis of the subject's medical history and the at least two physiological parameters whenever the degrees of severity Ds estimated for each physiological parameter Pp is comprised in a range of values R corresponding to a normal condition of the subject, wherein the analysis is performed by providing as input to a decision tree the subject's medical history and the at least two physiological parameters so as to output a recommendation to a user, said recommendation comprising at least one medicament and a dosage of said medicament.

According to one embodiment, the decision tree of the first calculation module is configured to provide three possible outputs:
a recommendation comprising the same medicament and dosage of the current prescription, whenever in a first preceding predefined time period at least one of the physiological values of the subject fall out of the range of values R corresponding to a normal condition and/or whenever in a second preceding predefined time period the current prescription has been provided to the subject by the user;
a recommendation comprising the same medicament of the current prescription and an updated dosage;
a recommendation comprising a medicament and a dosage different from the current prescription.

According to one embodiment, the system comprises a second calculation module configured to perform an analysis of the subject's medical history and the at least two physiological parameters whenever at least one of the degrees of severity Ds estimated for each physiological parameter Pp falls out of a range of values R corresponding to a normal condition of the subject, wherein the second calculation module is configured to output, based on the analysis, a recommendation to the user comprising at least a medicament and a dosage of said medicament.

In one embodiment, the recommendation outputted by the second calculation module is determined from a matrix configured to associate each combination of values of the physiological parameters to at least one medicament and its dosage.

According to one embodiment, the system further comprises a visualization module configured to displaying the recommendation together with a graphical representation of each measured physiological parameters Pp overtime, the current prescription of the user and a link to a medical history of the user. The displaying step help the user (i.e. physician) to choose the correct recommendation in a short time and to send a new prescription to the patient using the system. The prescription may be sent by others means as a mail or directly delivered at the user during an appointment with the practitioner for example.

The visualization module may be as well configured to displaying a graphical representation of the levels of computed physiological parameters (i.e. Estimated Glomerular Filtration Rate, etc.) resulting from the analysis the physiological parameters (i.e.: hemoglobin, potassium, creatinine, etc.).

In one example, the raw graph of creatinine measurement is displayed along with Estimated Glomerular Filtration Rate (computed from creatinine levels) and Worsening renal function, computed from current creatinine level and baseline creatinine levels.

In one embodiment, the visualization module is further configured to display a dashboard, said dashboard being configured to represent subject outputs obtained from the subject's history analysis module, the first calculation module and the second calculation module.

In one embodiment, the dashboard represents simultaneously subject outputs obtained from multiple subjects for the user.

More generally, the dashboard may be used to visualize for the user the recommendations and other pertinent data for at least a part of the patients for which the present system is used. The dashboard allows the physician to read all necessary information, for example in the form of a list. For example, the dashboard may be divided into three or more sections, wherein each section comprises the patients to a degree of severity Ds obtained severity estimation module. For example, the patients associated to the higher degree of severity Ds are displayed at the top of a list of patients. The recommendation for each patient has a color with respect to the degree of severity Ds of the patient. For example, the color red may be used for users that require medical attention, orange for users that have overdue action and green for users that do not need action. Other colors may be used as well.

For each patient, a column with a specific keyword of the situation may be provided allowing the physician to have a quick glance at the situation.

Form the global dashboard the user may access an individual patient page comprising useful information such as for example the values of the hematological parameter in the blood sample, the recommendation of the first or second calculation module and the previous prescription or clinician markers. The individual patient page is intended to facilitate the clinical-decision-making process of the physician.

The individual patient page may comprise data from the patient personal health record such as physiological parameters (renal function, congestion, potassium). For the renal function, a raw graph of creatinine measurement is displayed with the estimated glomerular filtration rate and the worsening renal function, computed from the current creatinine level and baseline creatinine contents. The three curves can be displayed upon clicking on the legend for example. The scale may adjust automatically. It is reminded that according to the invention, at least two curves are needed including hemoglobin. If the estimated glomerular filtration rate curve is displayed alone, graphic threshold for low and severely low worsening condition is displayed in two different colors (pink and red for example). A raw graph of hemoglobin measurement may be displayed along with the hemoglobin variation, computed from the current hemoglobin level and baseline hemoglobin contents. The three curves can be displayed upon clicking on the legend for example. According to one embodiment, at least two curves are displayed: the hemoglobin curve and the curve representing another physiological parameter as potassium or creatinine or other. The scale may adjust automatically. If the hemoglobin variation curve is displayed alone, the graphic threshold for congestion and decongestion is displayed in a color (red for example).

According to one embodiment, at least two curves are displayed: the potassium curve and a curve representing a physiological parameter as creatinine, hemoglobin or other.

A table format may be used for displaying the current prescriptions. For example, the table may comprise the name of the drugs prescribed along with the current dosage and the relevant target dose (set by the system for each drug and based on medical guidance). A link to a graphic display of the history of prescriptions is available.

The visualisation module further allows to visualise the output of the first or second calculation module, i.e. the recommendation. The recommendation may be presented as a text split in different relevant section with items or actions to investigate. This textual recommendation is displayed just above the physiological parameters, prescription section and above the prescription change form. It can be in any other visible place.

The visualisation module is further configured to allow the user to fill an input form. This form allows the physician to enter changes of dosage he might have decided.

The display is split in the six relevant categories of heart failure drugs (Angiotensin-converting enzyme Inhibitors/Angiotensin II Receptor Blockers/Angiotensin Receptor/Neprilysin Inhibitors, Mineralocorticoid Receptor Antagonists, Beta blockers, diuretics, potassium supplements, potassium binders). The display may be split in other categories concerning other drugs. These drug examples are in no way limiting the scope of the invention but just illustrations.

For each category of drug, the physician can fill a drop-down menu with a list of generic drug names, a free area to log the specific commercial name if required, the dosage, the frequency and the target dose. This fill box is automatically filled upon selection of a drug in the drop-down menu. It can be modified by the physician. In order to ease the modification of the current prescription, the form is automatically filled with the current prescription.

The present invention also relates to a computer program comprising instructions, which when the program is executed by a computer, causes the computer to carry out the steps of the method described above.

The computer program product to perform the method as described above may be written as computer programs, code segments, instructions or any combination thereof, for individually or collectively instructing or configuring the processor or computer to operate as a machine or special-purpose computer to perform the operations performed by hardware components. In one example, the computer program product includes machine code that is directly executed by a processor or a computer, such as machine code produced by a compiler. In another example, the computer program product includes higher-level code that is executed by a processor or a computer using an interpreter. Programmers of ordinary skill in the art can readily write the instructions or software based on the block diagrams and the flow charts illustrated in the drawings and the corresponding descriptions in the specification, which disclose algorithms for performing the operations of the method as described above.

The present invention also relates to a computer-readable storage medium comprising instructions that when executed by a computer, causes the computer to carry out the steps of the method.

According to one embodiment, the computer-readable storage medium is a non-transitory computer-readable storage medium.

Computer programs implementing the method of the present embodiments can commonly be distributed to users on a distribution computer-readable storage medium such as, but not limited to, an SD card, an external storage device, a microchip, a flash memory device, a portable hard drive and software websites. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While various embodiments have been described and illustrated, the detailed description is not to be construed as being limited hereto. Various modifications can be made to the embodiments by those skilled in the art without departing from the true spirit and scope of the disclosure as defined by the claims.

1.-21. (canceled)

22. A system for optimizing a treatment regimen of a subject, said subject suffering of heart failure with a risk of congestive heart failure or a risk of side effects due to heart failure medication, said system comprising:
a reception module configured to receive at least two physiological parameters, of which at least one being a hematological parameter obtained from an analysis of a blood sample of the subject;
a severity estimation module configured to estimate a degree of severity for each physiological parameter by comparing each physiological parameter to at least one predefined threshold and/or range of values;
a subject's history analysis module configured to analyze a subject's medical history, wherein the subject's medical history is retrieved from a database, said medical history comprising at least one of the following: clinical markers, the values of the at least two physiological parameters as a function of time, a current prescription of the user, at least one previous prescription and/or information related to former health condition;
a first calculation module configured to perform an analysis of the subject's medical history and the at least two physiological parameters whenever the degree of severity estimated for each physiological parameter is comprised in a range of values corresponding to a normal condition of the subject, wherein the analysis is performed by providing as input to a decision tree the subject's medical history and the at least two physiological parameters so as to output a recommendation to a user, said recommendation comprising at least one medicament and a dosage of said medicament; and
a second calculation module configured to perform an analysis of the subject's medical history and the at least two physiological parameters whenever at least one of the degrees of severity estimated for each physiological parameter falls out of a range of values corresponding to a normal condition of the subject, wherein the second calculation module is configured to output, based on the analysis, a recommendation to the user comprising at least a medicament and a dosage of said medicament.

23. The system according to claim 22, wherein the hematological parameter is measured daily.

24. The system according to claim 22, wherein the at least one hematological parameter is selected from the following list: hemoglobin, potassium or creatinine.

25. The system according to claim 22, wherein the at least one hematological parameter is the blood natriuretic peptide.

26. The system according to claim 22, wherein the at least one of the physiological parameters is selected from the following list: pulmonary artery pressure, lung impedance, weight, blood oxygen, blood pressure or electrocardiographic signal.

27. The system according to claim 22, wherein the first calculation module being configured to provide three possible outputs:
a recommendation comprising the same medicament and dosage of the current prescription, whenever in a first preceding predefined time period at least one of the physiological values of the subject fall out of the range of values corresponding to a normal condition and/or whenever in a second preceding predefined time period the current prescription has been provided to the subject by the user;
a recommendation comprising the same medicament of the current prescription and an updated dosage; and
a recommendation comprising a medicament and a dosage different from the current prescription.

28. The system according to claim 22, wherein, when at least one of the degrees of severity estimated for each physiological parameter fall out of the range of values corresponding to a normal condition of the subject, the recommendation outputted by the second calculation module is determined from a matrix configured to associate each combination of values of the physiological parameters to at least one medicament and its dosage.

29. The system according to claim 22, further comprising a visualization module configured to display the recommendation together with a graphical representation of each of at least two physiological parameters overtime.

30. The system according to claim 29, wherein the visualization module is further configured to display the current prescription of the user and a link to a medical history of the user.

31. The system according to claim 29, wherein the visualization module further configured to display a dashboard, the dashboard being configured to represent a subject identifier long with module outputs obtained from the subject's history analysis module, the first calculation module and the second calculation module.

32. The system according to claim 29, wherein the dashboard represents simultaneously subject outputs obtained from multiple subjects for the user.

33. A method for optimizing a treatment regimen of a subject, said subject suffering of heart failure with a risk of congestive heart failure or a risk of side effects due to heart failure medication, said method comprising the following steps:
obtaining at least two physiological parameters, of which at least one being a hematological parameter obtained from the analysis of a blood sample of the subject;
estimating a degree of severity for each physiological parameter by comparing each physiological parameter to at least one predefined threshold and/or at least one predefined range of values;
analyzing a subject's medical history, wherein the medical history is retrieved from a database, said medical history comprising at least one of the following: clinical markers, the values of the at least two physiological parameters as a function of time, a current prescription, at least one previous prescription and/or information related to former health condition;
if the degrees of severity estimated for each physiological parameter is comprised in a range of values corresponding to a normal condition of the subject, providing as input to a decision tree the subject's medical history and the at least two physiological parameters so as to output a recommendation to a user comprising at least a medicament and a dosage of said medicament; and
if at least one of the degrees of severity estimated for each physiological parameter is not comprised in a range of values corresponding to a normal condition of the subject, outputting to the user a recommendation comprising at least a medicament and a dosage of said medicament.

34. The method according to claim 33, wherein the at least one hematological parameter is selected from the following list: hemoglobin, potassium or creatinine.

35. The method according to claim 33, wherein the at least one of the physiological parameters is selected from the following list: pulmonary artery pressure, lung impedance, weight, blood oxygen, blood pressure or electroencephalographic signal.

36. The method according to claim 33, wherein the decision tree being configured to provide three possible outputs:
a recommendation comprising the same medicament and dosage of the current prescription, whenever in a first preceding predefined time period at least one of the physiological values of the subject fall out of the range of values corresponding to a normal condition and/or whenever in a second preceding predefined time period the current prescription have been provided to the subject by the user;
a recommendation comprising the same medicament of the current prescription and an updated dosage; and
a recommendation comprising a medicament and a dosage different from the current prescription.

37. The method according to claim 33, wherein when at least one of the degrees of severity estimated for each physiological parameter fall out of the range of values corresponding to a normal condition of the subject the recommendation is determined from a matrix configured to associate each combination of values of the physiological parameters to at least one medicament and its dosage.

38. The method according to claim 33, further comprising displaying the recommendation together with a graphical representation of each of at least two physiological parameters overtime, the current prescription of the user and a link to a medical history of the user.

39. The method according to claim 38, further comprising displaying a dashboard, the dashboard being configured to represent subject outputs obtained from the subject's history analysis module, the first calculation module and the second calculation module.

40. The method according to claim 38, wherein the dashboard represents simultaneously subject outputs obtained from multiple subjects for the user.

41. A computer program product for optimizing a treatment regimen of a user, the computer program product comprising instructions which, when the program is executed by a computer, causes the computer to carry out the steps of the method according to claim 33.

42. A computer-readable storage medium comprising instructions which, when the program is executed by a computer, causes the computer to carry out the steps of the method according to claim 33.
